(19) United States
(12) Patent Application Publication
(10) Pub. No.: US 2023/0256179 A1
(43) Pub. Date: Aug. 17, 2023

(54) DETERMINATION OF THE USAGE STATUS OF A DEVICE FOR THE ONE-TIME ADMINISTRATION OF A MEDICAMENT

(71) Applicant: BAYER AKTIENGESELLSCHAFT, Leverkusen (DE)

(72) Inventors: Markus HALM, Machern (DE); Armin HOSSEINI, Berlin (DE)

(21) Appl. No.: 18/001,929

(22) PCT Filed: Jun. 9, 2021
(86) PCT No.: PCT/EP2021/065413

§ 371 Date: Dec. 15, 2022

(30) Foreign Application Priority Data
Jun. 16, 2020 (EP) 20180180.0

(51) Int. Cl. A61M 5/50

(52) U.S. Cl. CPC A61M 5/5086; A61M 2205/502; A61M 2205/3515

(57) ABSTRACT

The invention relates to an apparatus for single administration of a medicinal product. The apparatus has a housing and a second component arranged for movement relative to the housing. During the administration of the medicinal product, the second component shifts from a first position relative to the housing into a second position relative to the housing. Fixed on the housing is a first magnetic element, and arranged on the moveable component is a second magnetic element. In the first position, the first magnetic element and the second magnetic element are close to one another (alternatively spatially separated from one another), and in the second position the first magnetic element and the second magnetic element are spatially separated from one another (alternatively close to one another), with the result that the magnetic field at the location of the first magnetic element is larger (alternatively smaller) when the moveable component is in the first position than when the moveable component is in the second position.

The invention also relates to a second apparatus having a magnetic field sensor for measuring the magnetic field at a predetermined position on the apparatus for single administration of a medicinal product, and for emitting a signal according to the measured strength of the magnetic field, and to a corresponding computer-implemented method.

The invention relates to an apparatus for single administration of a medicinal product for medical treatment. The invention relates in particular to a medicament dispensing apparatus for self-medication.

Apparatuses for single administration of a medicinal product include, for example, pre-filled syringes or cartridges. These are often combined with auto-injectors so that the patient is able to administer the medicinal product from the pre-filled syringe or cartridge independently. Auto-injectors come in an electronic version with motor or else in a mechanical version.

An important safety requirement in apparatuses for single administration of a medicinal product is identifying whether or not the apparatus has been used already. This is done for safety reasons to prevent only some of the contents having been used already or the contents being contaminated. This is also done because the patient himself perhaps no longer remembers whether or not he has already used the apparatus fully or partially, and it is not readily identifiable from the outside that usage has taken place.

Electronic auto-injectors are known that contain systems for tracking the usage of the auto-injector. These systems gather and save data relating to the time of use, duration of administration, quantity of the administered medicinal product, and possible further data. If applicable, the data is also analysed and communicated to further devices. Or auxiliary units are proposed, which are connected to the auto-injector in order to gather data and to assist the user possibly with the intake, for instance by a reminder function that emits a signal to the user in the event of a certain time without usage.

WO2019/032784 A1 discloses, for example, a medicament dispensing system for a pre-filled syringe having an adapter which is attached to the medicament dispensing system and reads out information from a magnetic sensor. The magnetic sensor is attached to the housing of the medicament dispensing system or to the adapter. The medicament dispensing system has a plunger, which pushes the medicament out of the pre-filled syringe. A permanent magnet is attached to this plunger.

WO2018/195270 A1 discloses a method for tracking the use of an auto-injector by a patient, and a corresponding external electronic adapter, which can accommodate the auto-injector. The electronic adapter has a magnetic field sensor, which detects the magnetic field of a magnetic element situated on a moveable component of the auto-injector.

All these systems have in common that they are operated electronically and by battery. The usage status is determined in some cases by means of magnetic field sensors which measure a different magnetic field depending on whether a movement of two components relative to each other has taken place. If a movement has taken place, it is assumed that the system for the administration has already been used.

A trend in auto-injectors, for reasons of hygiene, are single-use auto-injectors that are disposed of after being used once. In order to conserve resources, single-use auto-injectors should not have any electronic components. Nevertheless, the safety requirement that the user can identify the status of usage of the system should also still be satisfied here.

The object of the present invention is to provide a system for identifying the usage status for apparatuses for single administration of a medicinal product, which system shall not have any electronic components.

The object according to the invention is achieved by an apparatus for single administration of a medicinal product. The apparatus may be a pre-filled syringe, or may be an auto-injector into which is inserted a cartridge or a pre-filled syringe, each of which may also be needleless. The apparatus according to the invention may also be a needleless syringe or a portable device such as a patch pump. Also conceivable are apparatuses consisting of a syringe and an auxiliary element or adapter. What is crucial is that in each case there is a first component and a second component, and the second component can move relative to the first component. The first component is called a “housing” below. The term “housing” includes, for example, a syringe body, an auxiliary element attached to the syringe body, or an auto-injector housing suitable for accommodating a pre-filled syringe or a cartridge (each optionally also needleless). The term “housing” also includes the housing of other mechanical injection systems. The second, moveable component may consequently be a syringe plunger or the plunger of an auto-injector. Also eligible as moveable components are further moveable elements of an auto-injector, for instance a drive spring or a needle shield, provided these elements experience a sufficiently large spatial displacement relative to the housing during administration of a medicinal product.

The apparatus according to the invention for single administration of a medicinal product has a housing and a second component arranged for movement relative to the housing. During the administration of the medicinal product, the second component shifts from a first position relative to the housing into a second position relative to the housing. Fixed on the housing is a first magnetic element, and arranged on the moveable component is a second magnetic element. In the first position the first magnetic element and the second magnetic element are close to one another, and in the second position the first magnetic element and the second magnetic element are spatially separated from one another, with the result that the magnetic field at the location of, or in the near field of, the first magnetic element is larger when the moveable component is in the first position than when the moveable component is in the second position.

Another embodiment of the apparatus according to the invention for single administration of a medicinal product has a housing and a second component arranged for movement relative to the housing. During the administration of the medicinal product, the second component shifts from a first position relative to the housing into a second position relative to the housing. Fixed on the housing is a first magnetic element, and arranged on the moveable component is a second magnetic element. In the first position the first magnetic element and the second magnetic element are spatially separated from one another, and in the second position the first magnetic element and the second magnetic element are close to one another, with the result that the magnetic field at the location of, or in the near field of, the first magnetic element is smaller when the moveable component is in the first position than when the moveable component is in the second position.

Each magnetic element produces a magnetic field. The strength M of magnetic fields decreases with increasing distance R from its origin according to the relationship M(R) ∼ 1/R². The magnetic field strength at a location is composed of the magnetic field strengths of all the sources of magnetic fields acting at this location. The magnetic field sources situated farther away, however, make little to practically no contribution to the total magnetic field strength compared with the magnetic field sources situated close to the location. Therefore a significantly higher magnetic field is measured at a location, or in its near field, at which two magnetic field sources are situated close to one another than if only one of the two sources is situated there, and the second source is situated at a location lying further away.

Eligible as the first and second magnetic element for the apparatus according to the invention are, for example, a permanent magnet or magnetic particles, which in each case are attached to the surface of the housing or of the moveable component, are enclosed in or between housing parts or parts of the moveable component, are integrated in the material of the housing or of the moveable component, or are part of a label applied on the housing or the moveable component. Preferably, the first and second magnetic elements have the same magnetic characteristics, so for instance are both permanent magnets made of the same material (NdFeB, Bismanol, ferrites) having the same magnetic field strength.

The measurement of the magnetic field strength at a predetermined position on the apparatus according to the invention is performed by means of a magnetic field sensor, which is not part of the apparatus for single administration of the medicinal product. Typical magnetic field sensors are Hall sensors. The magnetic field sensor is part of a separate device that has the functionalities of a computer. The position at which, or in the proximity of which, the magnetic field strength is measured is preferably indicated externally on the housing. Another possible option for the indication is advice on the local region of the measurement via an interface of the separate device for the user (graphical user display or the like) or via operating instructions.

The separate device has at least the following features:
a. a magnetic field sensor connected to a processor, wherein the processor is connected to a memory and to an interface with the user or to a communications interface;
b. in the memory are stored value ranges for magnetic fields which correspond to a strength range of a magnetic field from a single magnetic element, and are stored value ranges for magnetic fields which correspond to a strength range of the total magnetic field from two magnetic elements;
c. each of the two value ranges in the memory is associated with a usage status;
d. the processor is programmed such that it compares a measured value from the magnetic sensor with the stored value ranges for magnetic fields, and in the event that the measured value coincides with one of the stored value ranges, emits via the interface with the user or via the communications interface a signal that corresponds to the usage status associated with the coincident value range.

The measured value being coincident with a stored value range means that the measured value lies on, or within, the limits of the value range. The value range may also consist of one or more single values.

The separate device may be a conventional smartphone, tablet computer or a smartwatch etc. as long as it has a magnetic field sensor, for instance a Hall sensor. This is the case, for example, if the device has a compass function. It is well known that devices such as a smartphone or smartwatch comprise the functionalities of a computer as standard. Via the interface with the user, for instance via a display, icon, loudspeaker, audio output, an optical, visual, haptic (vibration) signal corresponding to the “used” or “unused” status can be given to the user. Via the communications interface, a signal can be sent to a further digital (terminal) device. The processor can be programmed also via an application, which is loaded onto the separate device.

The invention also relates to a computer-implemented method,

comprising the steps of:
reading out a measured value from a magnetic field sensor;
comparing the measured value with stored value ranges for magnetic fields which correspond to the strength or a strength range of a magnetic field from a single magnetic element, and which correspond to the strength or a strength range of the total magnetic field from two magnetic elements, wherein each of the two stored value ranges in the memory is associated with a usage status;
in the event that the measured value coincides with one of the stored value ranges, emitting via an interface with the user or via a communications interface a signal that corresponds to the usage status associated with the coincident value range.

The invention also relates to a computer program product comprising the commands which, when executed by a computer connected to a magnetic field sensor, cause this computer to perform the following method steps:
reading out a measured value from a magnetic field sensor;
comparing the measured value with stored value ranges for magnetic fields which correspond to the strength or a strength range of a magnetic field from a single magnetic element, and which correspond to the strength or a strength range of the total magnetic field from two magnetic elements, wherein each of the two stored value ranges in the memory is associated with a usage status;
in the event that the measured value coincides with one of the stored value ranges, emitting via an interface with the user or via a communications interface a signal that corresponds to the usage status associated with the coincident value range.

The computer program product may be an application for a smartphone, tablet computer or a smartwatch, etc.

### FIGURES AND EXAMPLES

The invention is explained more fully below in conjunction with preferred embodiments and with reference to the drawings.

FIG. 1 Auto-injector in the unused state

FIG. 2 Auto-injector in the used state

FIG. 3 Auto-injector in the unused state including magnetic elements in a first embodiment

FIG. 4 Auto-injector in the used state including magnetic elements in a first embodiment

FIG. 5 Auto-injector in the unused state including magnetic field lines in a first embodiment

FIG. 6 Auto-injector in the used state including magnetic field lines in a first embodiment

FIG. 7 Auto-injector in the unused state including magnetic elements in a second embodiment

FIG. 8 Auto-injector in the used state including magnetic elements in a second embodiment

FIG. 9 Auto-injector in the unused state including magnetic field lines in a second embodiment

FIG. 10 Auto-injector in the used state including magnetic field lines in a second embodiment

FIG. 11 Syringe in the unused state

FIG. 12 Syringe in the used state

FIG. 13 Syringe in the unused state including magnetic elements in a first embodiment

FIG. 14 Syringe in the used state including magnetic elements in a first embodiment

FIG. 15 Syringe in the unused state including magnetic field lines in a first embodiment

FIG. 16 Syringe in the used state including magnetic field lines in a first embodiment

FIG. 17 Syringe in the unused state including magnetic elements in a second embodiment

FIG. 18 Syringe in the used state including magnetic elements in a second embodiment

FIG. 19 Syringe in the unused state including magnetic field lines in a second embodiment

FIG. 20 Syringe in the used state including magnetic field lines in a second embodiment

### REFERENCE SIGNS

100 - auto-injector
101 - housing
102 - proximal end
104 - distal end
110 - plunger rod
120 - viewing window
200 - ready-to-use syringe
201 - syringe body
202 - open end
203 - flange
204 - syringe shoulder
206 - syringe cone
208 - cannula
210 - syringe plunger rod
212 - stopper
214 - plunger flange
215 - finger flange
310 - first magnetic element
320 - second magnetic element
330 - single magnetic field
340 - double magnetic field
350 - indicator
351 - measurement position

Some preferred embodiments of the invention are presented in the following examples. It should be stressed, however, that the invention is not restricted thereto, but may be embodied in other ways within the subject matter defined in the claims.

For the sake of clarity, the figures are schematic and simplified. They show only details that are essential to the invention, while other details have been omitted. The same reference numbers are used throughout for identical or equivalent parts.

### EXAMPLE 1

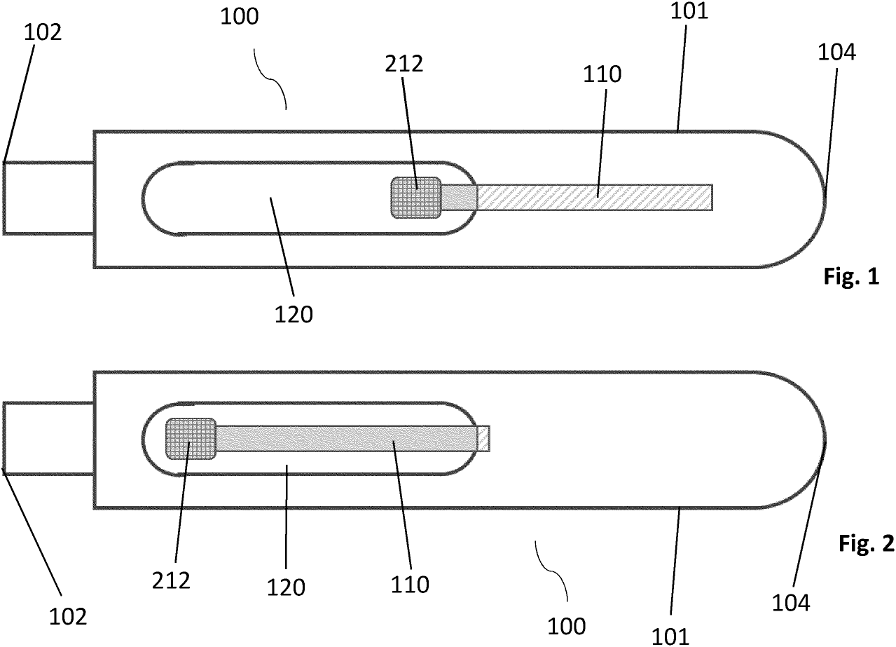

FIGS. 1 and 2 show an auto-injector 100 having a housing 101, which has a proximal end 102 and a distal end 104. A cartridge with cannula or ready-to-use/pre-filled syringe (neither shown) can be inserted into the housing 101. In order to administer the drug out of the ready-to-use syringe or cartridge, a plunger rod 110, which is mounted in the housing 101 for movement parallel to the longitudinal axis of the housing, can be displaced from a starting position (FIG. 1) in the proximal direction into an end position (FIG. 2). The plunger rod 110 is in contact with a stopper 212, and, during the administering, pushes on the stopper 212 of the ready-to-use syringe or the cartridge. The drug is thereby pressed out of the ready-to-use syringe or cartridge in the proximal direction. A portion of the plunger rod 110 can be seen through a viewing window 120 in the housing 101. A striped pattern indicates the portion of the plunger rod 110 that cannot be seen through the viewing window 120. The ready-to-use syringe or cartridge is not shown, but only the stopper 212 as the interface between the plunger rod 110 of the auto-injector 100 and the ready-to-use syringe or cartridge.

### Example 1A

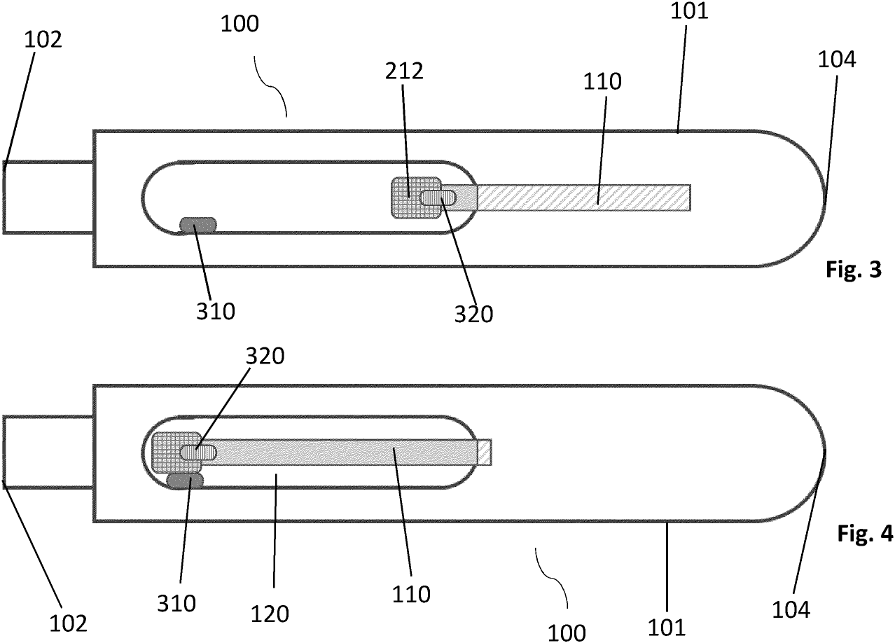

FIGS. 3 and 4 show the auto-injector 100 having a first magnetic element 310 and a second magnetic element 320. The first magnetic element 310 is situated on the internal wall of the housing 101 in the region of the proximal end of the viewing window 120. The second magnetic element 320 is attached to the plunger rod 110 in the transition region to the stopper 212. The plunger rod 110 having the second magnetic element can be seen in the region of the distal end of the viewing window 120. In FIG. 3, the auto-injector is unused, i.e. the plunger rod 110 and the stopper 212, and hence the magnetic element 320. are situated away from the first magnetic element 310 in a more distal position. In FIG. 4, the auto-injector has been actuated, and the plunger rod 110 has moved from a more distal position into a more proximal position. Now the plunger rod 110 and the stopper 212, and hence the magnetic element 320, are situated close to the first magnetic element 310.

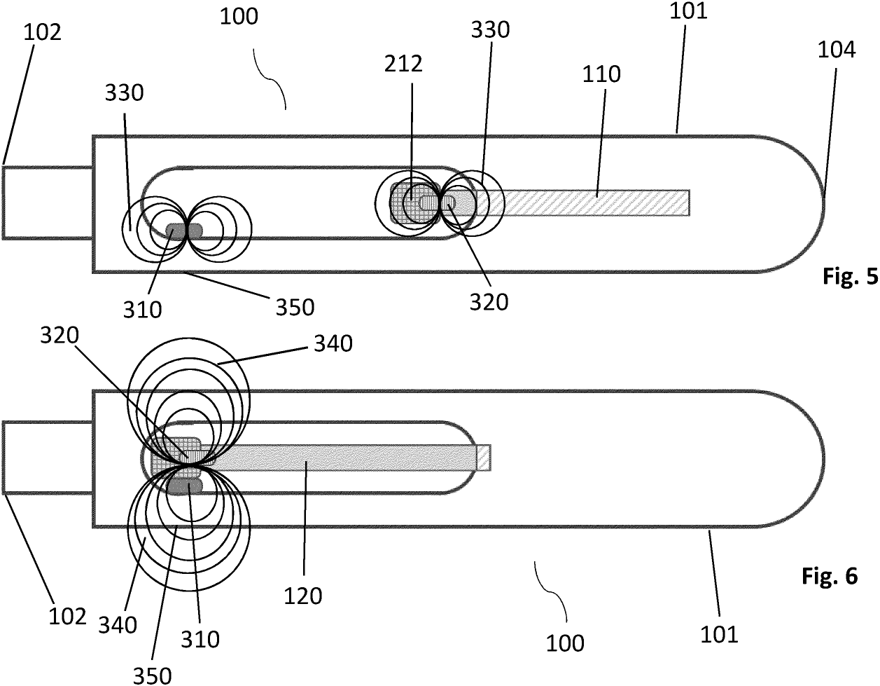

FIGS. 5 and 6 use magnetic field lines to show the magnetic fields 330, 340 associated with the positions adopted by the magnetic elements 310, 320 in FIG. 3 and FIG. 4. It is indicated schematically that the strength of the magnetic field of a single, separate magnetic element 310, 320 (FIG. 5) is smaller, or falls below a certain value at a shorter distance from the magnetic element 310, 320, than the magnetic field strength of two magnetic elements situated close to one another (FIG. 6). This effect is employed in determining the “used” or “unused” state .

In the present example, the device having the magnetic field sensor is a smartphone. The smartphone contains a compass and is thereby suitable for detecting magnetic fields. An application that has been loaded into the smartphone controls the measurement of the magnetic field strength by the magnetic field sensor and the processing of the measured values.

The measurement of the magnetic field strength is performed at, or in the proximity of, an indicator 350 on the housing 101 of the auto-injector 100. The first magnetic element 310 is situated here on the inside of the housing 101. The smartphone display is brought into the region of the indicator 350 on the housing 101 or into the vicinity of the housing 101 of the auto-injector 102, and the value of the magnetic field Mₓ is detected. The indicator 350 advises the user as to exactly where the smartphone with its display is meant to be placed on, or close to, the housing 101. It is important to perform every measurement of the magnetic field strength within a certain proximity of the location of the first magnetic element 310 so that the measurements are comparable. Instead of the display, another predetermined part of the smartphone can also be brought into the proximity of the indicator 350 for the measurement. The indicator 350 on the housing 101 may be, for example, a marker on the outside of the housing 101 or an adhesively attached label.

By means of the application, value ranges [M1.... M₂] for magnetic fields which correspond to the strength range of the magnetic field of the magnetic element 310 have been stored in the memory of the smartphone. A strength range because the measured values can have a certain spread depending on the distance of the magnetic field sensor from the magnetic element 310. Similarly, in the memory of the smartphone have been stored value ranges [M₃.... M₄] for magnetic fields which correspond to the strength range of the summated magnetic field of the magnetic elements 310 and 320 when these are situated close to one another.

The processor of the smartphone is programmed by the application to compare the measured value Mx from the magnetic field sensor with the stored value ranges [M₁.... M₂] and [M₃.... M₄] for magnetic fields. If measured value Mx lies in one of the stored value ranges [M₁.... M₂] or [M₃.... M₄], the smartphone emits a corresponding signal to the user. For example, on the display can appear the words “Already used” if the measured value Mx lies in the value range [M₃.... M₄], or the word “Unused” if the measured value Mₓ lies in the value range [M₁.... M₂]. Alternatively, the smartphone can start to vibrate as a sign for “used” if the measured value Mx lies in the value range [M₃.... M₄], and can make a triad sound if the measured value Mx lies in the value range [M₁.... M₂]. Numerous variants are conceivable here.

### Example 1B

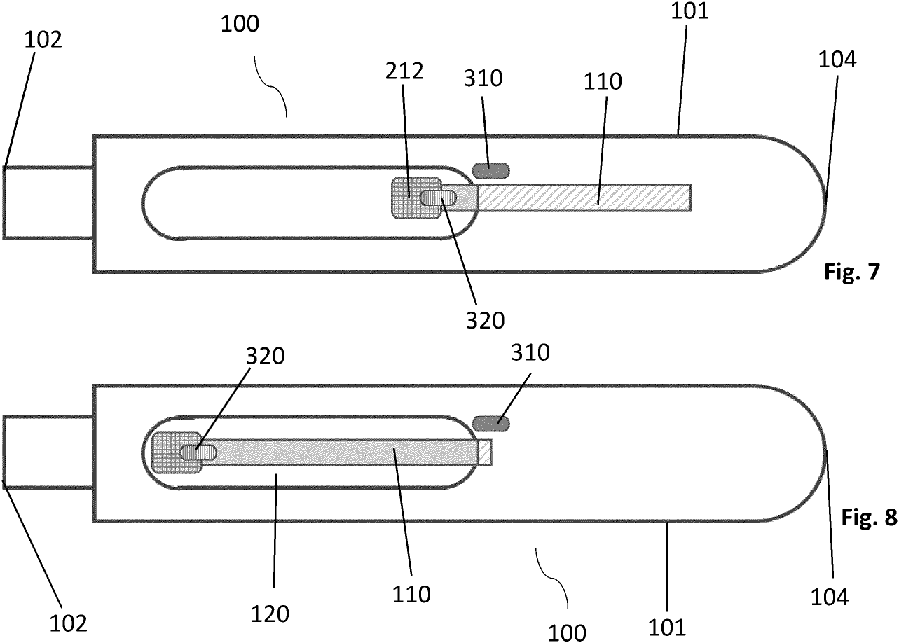

FIGS. 7 and 8 show the auto-injector 100 having an alternative arrangement of the first magnetic element 310 and the second magnetic element 320. The first magnetic element 310 is situated on the internal wall of the housing 101 in the region of the distal end of the viewing window 120. The second magnetic element 320 is attached to the plunger rod 110 in the transition region to the stopper 212. The plunger rod 110 having the second magnetic element can be seen in the region of the distal end of the viewing window 120. In FIG. 7, the auto-injector is unused, i.e. the plunger rod 110 and the stopper 212, and hence the magnetic element 320, are situated close to the magnetic element 310. In FIG. 8, the auto-injector has been actuated, and the plunger rod 110 has moved from a more distal position into a more proximal position. Now the plunger rod 110 and the stopper 212, and hence the magnetic element 320, are situated away from the first magnetic element 310.

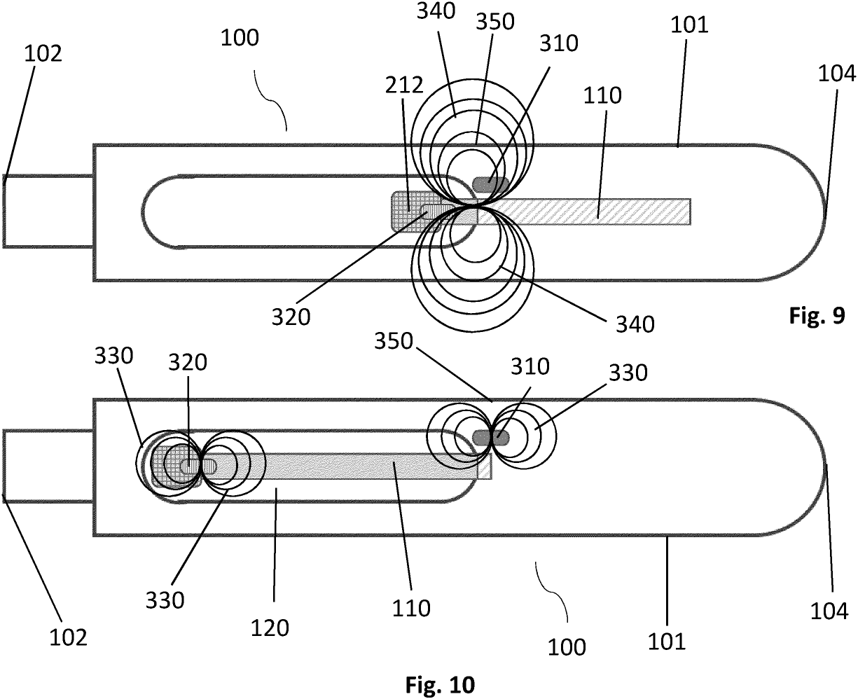

FIGS. 9 and 10 use magnetic field lines to show the magnetic fields 330, 340 associated with the positions adopted by the magnetic elements 310, 320 in FIG. 7 and FIG. 8. It is indicated schematically that the strength of the magnetic field of one single, separate magnetic element 310, 320 (FIG. 10) is smaller, or falls below a certain value at a shorter distance from the magnetic element 310, 320, than the magnetic field strength of two magnetic elements situated close to one another (FIG. 9). This effect is employed in determining the “used” or “unused” state .

The measurement and processing of the magnetic field strength is performed as in Example 1A by means of a smartphone. In this exemplary embodiment, the identifier 350 is situated in the region of the distal end of the viewing window 120, where the first magnetic element 310 is situated on the inside of the housing 101. Unlike example 1A, in this exemplary embodiment, the smartphone application is programmed such that a signal corresponding to the “used” usage status is emitted to the user if the measured value Mx lies in the value range [M₁.... M₂]. A signal corresponding to the “unused” usage status is emitted if the measured value Mx lies in the value range [M₃.... M₄].

### EXAMPLE 2

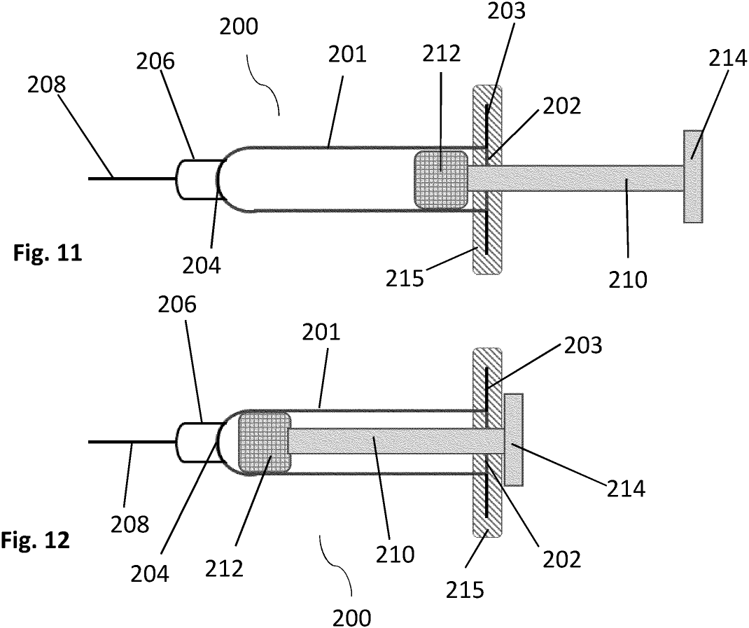

FIGS. 11 to 20 show a further embodiment of the invention. Instead of the auto-injector 100, the apparatus for single administration of a medicinal product is a ready-to-use syringe 200.

As shown in FIGS. 11 and 12, the ready-to-use syringe 200 has a syringe body 201 having an open end 202. Around the open end is a flange 203. Opposite the open end 203, the syringe body 201 has the syringe shoulder 204. At the syringe shoulder 204 is situated the syringe cone 206 as an attachment for a cannula 208. Inside the syringe body 201 is the stopper 212, which seals the fluid-holding volume inside the syringe body 201 with respect to the open end 202, and is connected to the syringe plunger rod 210, which protrudes through the open end 202 from the region of the syringe body 201. The syringe plunger rod 210 terminates outside the syringe body 201 with a plunger flange 214. Fitted to the plunger flange 214 is also a finger flange 215, which makes the syringe easier to operate for the user.

### Example 2A

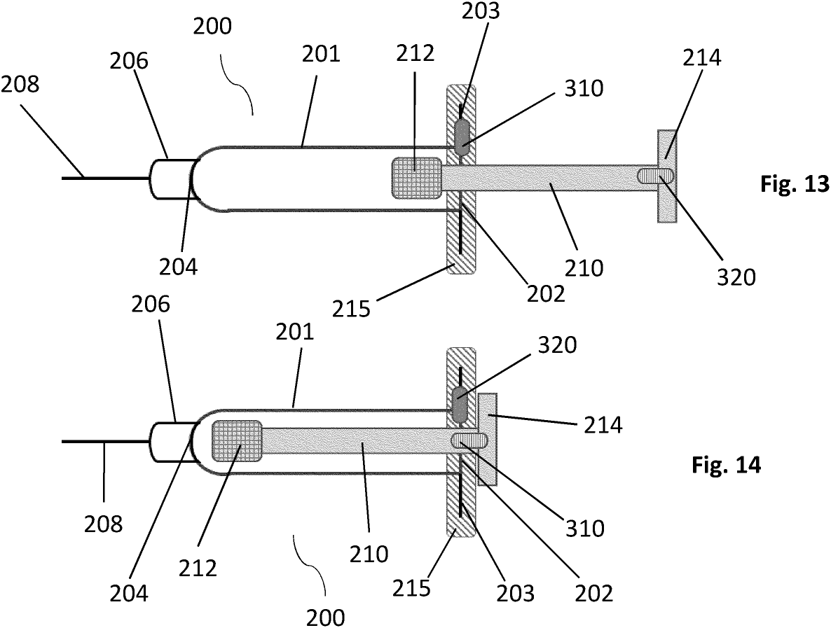

FIGS. 13 and 14 show the ready-to-use syringe 200 having a first magnetic element 310 and a second magnetic element 320. The first magnetic element 310 is situated at the flange 203. The second magnetic element 320 is attached to the syringe plunger rod 210 in the transition region to the plunger flange 214. In FIG. 13, the ready-to-use syringe is unused, i.e. the syringe plunger rod 210 is withdrawn from the inside of the syringe body 201, and the stopper 212 is situated at the open end 202. The plunger flange 214, and hence the magnetic element 320, is situated away from the first magnetic element 310. In FIG. 14, the ready-to-use syringe 200 has been used, and the syringe plunger rod 210, with the stopper 212, has been moved towards the syringe shoulder 204. The flange 214, with the magnetic element, is now situated close to the flange 203 having the second magnetic element 320.

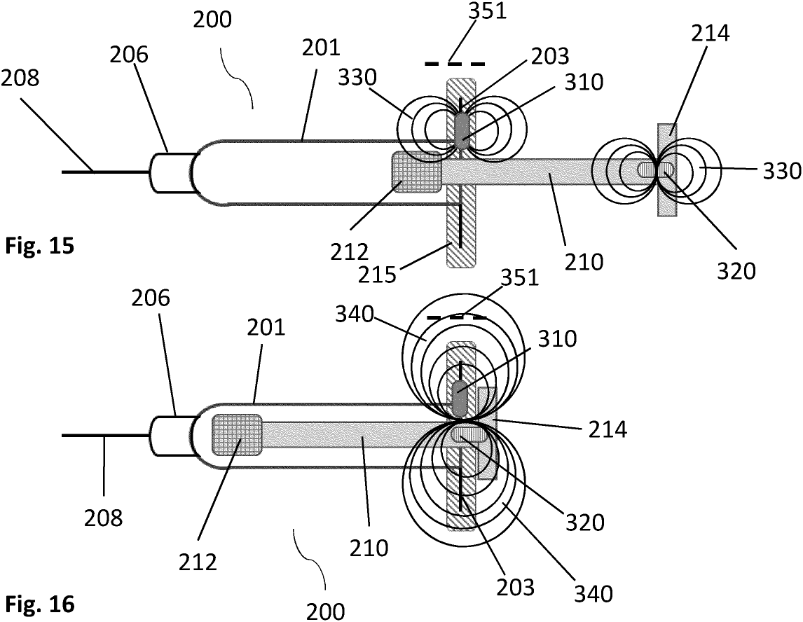

FIGS. 15 and 16 use magnetic field lines to show the magnetic fields 330, 340 associated with the positions adopted by the magnetic elements 310, 320 in FIG. 13 and FIG. 14. It is indicated schematically that the strength of the magnetic field of a single, separate magnetic element 310, 320 (FIG. 15) is smaller, or falls below a certain value at a shorter distance from the magnetic element 310, 320, than the magnetic field strength of two magnetic elements situated close to one another (FIG. 16). This effect is employed in determining the “used” or “unused” state .

The measurement and processing of the magnetic field strength is performed as described in Example 1A by means of a smartphone. In this exemplary embodiment, the measurement position 351 is situated in the vicinity of the flange, as indicated by the dashed line. Alternatively, the measurement position 203 could also be situated directly on the flange 203. Determining the usage status of a ready-to-use syringe 200 by means of a magnetic field sensor is also expedient even if it can be seen from the ready-to-use syringe 200 that it has been used. For instance the usage data can thus be captured immediately electronically and used for tracking the medicament dispensing.

### Example 2B

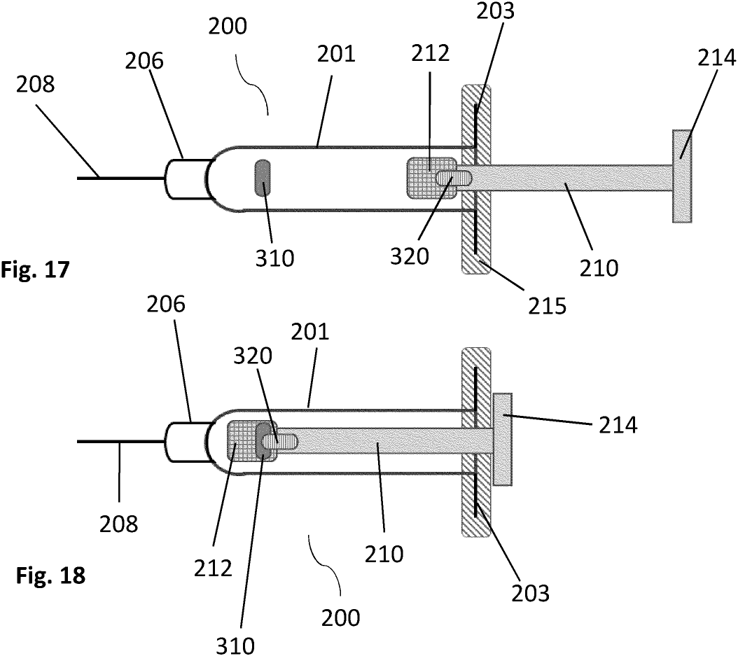

FIGS. 17 and 18 show the ready-to-use syringe 200 having an alternative arrangement of the first magnetic element 310 and the second magnetic element 320. The first magnetic element 310 is situated in the region of the syringe shoulder 204 on the external wall of the syringe body 201. The second magnetic element 320 is attached to the syringe plunger rod 210 in the transition region to the stopper 212. In FIG. 17, the ready-to-use syringe is unused, i.e. the syringe plunger rod 210 is withdrawn from the inside of the syringe body 201, and the stopper 212 is situated at the open end 202. The stopper 212, and hence the magnetic element 320, is situated away from the first magnetic element 310. In FIG. 18, the ready-to-use syringe 200 has been used, and the syringe plunger rod 210, with the stopper 212, has been moved towards the syringe shoulder 204. The stopper 212, with the magnetic element 320. is now situated close to the second magnetic element 320 at the syringe shoulder 204.

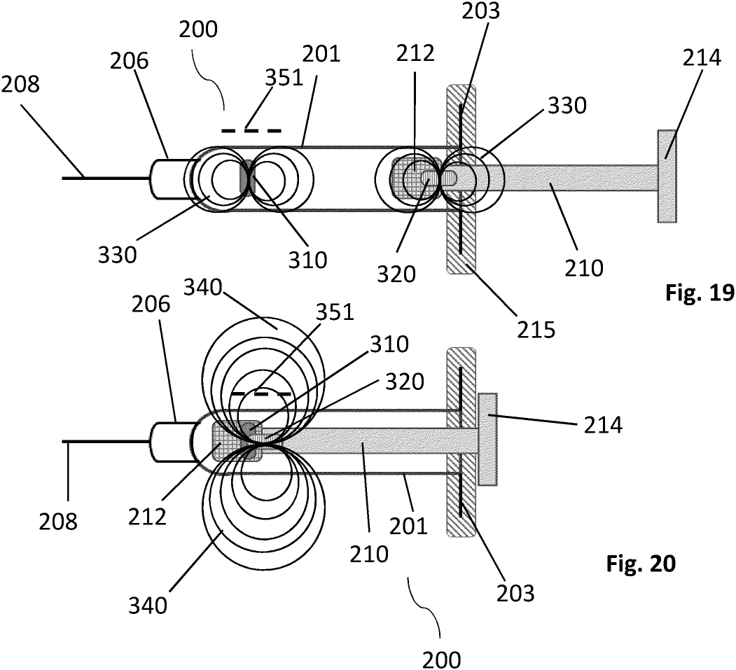

FIGS. 19 and 20 use magnetic field lines to show the magnetic fields 330, 340 associated with the positions adopted by the magnetic elements 310, 320 in FIG. 17 and FIG. 18. It is indicated schematically that the strength of the magnetic field of a single, separate magnetic element 310, 320 (FIG. 19) is smaller, or falls below a certain value at a shorter distance from the magnetic element 310, 320, than the magnetic field strength of two magnetic elements situated close to one another (FIG. 20). This effect is employed in determining the “used” or “unused” state .

The measurement and processing of the magnetic field strength is performed as described in Example 1A by means of a smartphone. In this exemplary embodiment, the measurement position 351 is situated in the region of the outlet end 204 in the vicinity of the external wall of the syringe body 201 but not directly on the external wall, as indicated by the dashed line. Alternatively, the measurement position 203 could also be situated directly on the housing 201. Determining the usage status of a ready-to-use syringe 200 by means of a magnetic field sensor is also expedient even if the ready-to-use syringe 200 is visible per se, and it could also be seen from the position of the syringe plunger rod 210 whether or not the ready-to-use syringe 200 has been used. By means of the described procedure, the usage data can be captured immediately electronically and used for tracking the medicament dispensing. The described procedure can also offer advantages to the visually impaired.

1. An apparatus for single administration of a medicinal product having a housing and a component arranged for movement relative to the housing,
wherein during the administration of the medicinal product, the moveably arranged component shifts from a first position relative to the housing into a second position relative to the housing,
wherein fixed on the housing is a first magnetic element, and arranged on the moveable component is a second magnetic element, and
in the first position, the first magnetic element and the second magnetic element are close to one another, and in the second position the first magnetic element and the second magnetic element are spatially separated from one another, with the result that the magnetic field at the location of the first magnetic element is larger when the moveable component is in the first position than when the moveable component is in the second position.

2. An apparatus for single administration of a medicinal product having a housing and a component arranged for movement relative to the housing,
wherein during the administration of the medicinal product, the moveably arranged component shifts from a first position relative to the housing into a second position relative to the housing,
wherein fixed on or in the housing is a first magnetic element, and arranged on the moveable component is a second magnetic element, and
in the first position, the first magnetic element and the second magnetic element are spatially separated from one another, and in the second position the first magnetic element and the second magnetic element are close to one another, with the result that the magnetic field at the location of the first magnetic element is smaller when the moveable component is in the first position than when the moveable component is in the second position.

3. The apparatus according to claim 1, characterized in that the housing is a syringe body, and the moveable component is a syringe plunger or a stopper.

4. The apparatus according to claim 1, characterized in that the housing is an auxiliary element attached to the syringe body, and the moveable component is a syringe plunger or a stopper, and the auxiliary element is a finger flange.

5. The apparatus according to claim 1, characterized in that the housing is an auto-injector housing suitable for accommodating a pre-filled syringe or cartridge, and the moveable component is a plunger rod, a drive spring or a needle shield.

6. The apparatus according to claim 1, characterized in that the first magnetic element is a permanent magnet or magnetic particles, which are attached to the surface of the housing, are enclosed in or between housing parts, are integrated in the material of the housing, or are part of a label applied on the housing.

7. The apparatus according to claim 1, characterized in that the second magnetic element is a permanent magnet or magnetic particles, which are attached to the surface of the moveable component, are enclosed in or between parts of the moveable component, are integrated in the material of the moveable component, or are part of a label applied on the moveable component.

8. The apparatus according to claim 1, characterized in that the location of the first magnetic element is identified externally on the housing.

9. An apparatus having a magnetic field sensor connected to a processor, wherein the processor is connected to a memory and to an interface with the user or to a communications interface,
wherein in the memory are stored value ranges for magnetic fields which correspond to a strength range of a magnetic field from a single magnetic element, and are stored value ranges for magnetic fields which correspond to a strength range of the total magnetic field from two magnetic elements, and in addition each of the value ranges is associated with a usage status, and
the processor is programmed such that it compares measured data from the magnetic field sensor with the stored value ranges for magnetic fields, and in the event that the measured data coincides with one of the stored value ranges, emits via the interface with the user or via the communications interface a signal that corresponds to the usage status associated with the coincident value range.

10. The apparatus according to claim 9, characterized in that the apparatus is a smartphone, a smartwatch or a tablet computer.

11. A computer-implemented method comprising the steps of:
a. reading out a measured value from a magnetic field sensor;
b. comparing the measured value with stored value ranges for magnetic fields which correspond to the strength or a strength range of a magnetic field from a single magnetic element, and which correspond to the strength or a strength range of the total magnetic field from two magnetic elements, wherein each of the two stored value ranges in the memory is associated with a usage status; and
c. in the event that the measured value coincides with one of the stored value ranges, emitting via an interface with the user or via a communications interface a signal that corresponds to the usage status associated with the coincident value range.

12. A computer program product comprising commands which, when executed by a computer connected to a magnetic field sensor, cause this computer to perform the following method steps:
a. reading out a measured value from the magnetic field sensor;
b. comparing the measured value with stored value ranges for magnetic fields which correspond to a strength range of a magnetic field from a single magnetic element, and correspond to a strength range of the total magnetic field from two magnetic elements, wherein each of the two stored value ranges in the memory is associated with a usage status; and
c. in the event that the measured value coincides with one of the stored value ranges, emitting via an interface with the user or via a communications interface a signal that corresponds to the usage status associated with the coincident value range.

13. The computer program product according to claim 12, characterized in that it is an application for a smartphone, a smartwatch or a tablet computer.
